(19) United States
(12) Patent Application Publication
(10) Pub. No.: US 2004/0009921 A1
(43) Pub. Date: Jan. 15, 2004

(54) Methods and compositions for the treatment of cancer

(75) Inventors: Jia-Hao Xiao, Irvine, CA (US); Corine Ghosn, Lake Forest, CA (US); Roshantha A. Chandraratna, Laguna Hills, CA (US)

Correspondence Address: Carlos A. Fisher, ALLERGAN, INC., T2-7H, 2525 Dupont Drive, Irvine, CA, 92612, US

(73) Assignee: Allergan, Inc.

(21) Appl. No.: 10/602,350

(22) Filed: Jun. 23, 2003

Related U.S. Application Data
(60) Provisional application No. 60/390,945, filed on Jun. 24, 2002.

(51) Int. Cl. A61K 38/17; A61K 48/00

(52) U.S. Cl. 514/12; 514/44; 424/93.2

(57) ABSTRACT

Methods and compositions for treatment of cancer and other proliferative diseases comprising administration of RXR nuclear receptor protein and an agonist thereof. In other aspects, the present application is drawn to methods of screening compounds for RXR agonist activity comprising determining whether a test compound stimulates the degradation of B-catenin.

### BACKGROUND OF THE INVENTION

The genetic basis of cancer has been established over the last 40 years. Initial findings constituted circumstantial evidence correlating human and animal exposure to mutatgenic agents with increased incidence of cancer. With the advent of the tools of molecular biology it was discovered that various and diverse factors involved in cell growth, and those genes and gene products that encode and/or regulate the expression of such factors may all play a part in the transformation of normal somatic cells into cells undergoing abnormal and largely uncontrolled cell division. The mutation of genes encoding such factors or the regulatory genes controlling their transcription, translation, post-translational modification, activity and/or turnover may result in the transformation of a somatic cell into a transformed cell.

Among those genes that have been identified as potential oncogenes, is the multifunctional protein β-catenin. β-catenin has been identified is a key player in regulating cell-cell adhesion, cell proliferation and motility. It is also a signal transducer in the Wnt signaling pathway in embryo development. In normal adult cells, β-catenin forms a complex with a tumor suppressor protein, E-cadherin; this complex is a factor in the simulation of cell-cell adhesion and inhibits cell growth. However, when not complexed with E-cadherin, β-catenin can play a part in the transcriptional activation of genes involved in cell proliferation and motility and has been associated with the development of various cancers, particularly, though not exclusively, colon cancer.

In normal cells, the free form of β-catenin is efficiently degraded through formation of a complex with the APC tumor suppressor and other proteins including axin and the serine/threonine kinase GSK-3β. GSK-3β phosphrylates APC, β-catenin and axin; phosphorylation of β-catenin results in its targeting for degradation by way of a ubiquitin-mediated proteosomeal pathway.

Somatic mutation in the β-catenin-interacting motifs of E-cadherin or APC (see e.g., Chung, D., Gastroenterology 119:854-865 (2000)), or in the APC-interacting motifs of β-catenin itself (see e.g., Harada et al., EMBO J. 18:5931-5942 (1999)), or abnormal activation of the Wnt signaling pathway can result in disruption of the APC:β-catenin complex and liberation and accumulation of the free cytoplasmic form of β-catenin. Excess β-catenin is translocated to the cell nucleus where it binds with transcription factors of the LEF/TCF pathway, such as Tcf-4, to up-regulate target genes including c-myc, cyclin D1, PPARδ and c-jun through binding of TCF-response elements. These genes appear to be critical for the proliferation and transformation of colonic epithelial cells. Accordingly, in human adults dysregulation of and/or mutations in β-catenin are believed to be not only one of the leading causes in colorectal cancer but also be involved in many other cancer types such as gastric, hepatocellular/hepatoblastoma, ovarian, endometrial prostate, kidney, melanoma, and thyroid cancers.

It has been suggested that retinoic acid participates in the regulation of the β-catenin-LEF/TCF signaling pathway. Retinoic acid was shown to decrease the degree of transcription of a LEF/TCF reporter gene in retinoid-sensitive APC mutant colon cancer (Caco-2) cells. This finding, as well as experiments using a transcriptionally active, non-ubiquitinable mutant of β-catenin suggests that RA can inhibit β-catenin transcriptional activity by a pathway other than that mediated by APC. Easwaran et al., Current Biology 9:1415-1418 (1999). These investigators also report that in vitro transcribed and translated RAR and RXR proteins interacted slightly with a GST-β-catenin fusion protein. The interaction of RAR, but not of RXR, was reportedly increased with the addition of retinoic acid; the presence of RXR in this reaction did not inhibit or stimulate β-catenin-RAR interactions.

Experiments conducted in human keritinocytes suggest that the regulation of the retinoic acid receptors (RARs) and retinoid X receptors (RXRs) is also accomplished in part by degradation of these proteins in vivo. Dimerization of RARγ with RXRα appears to be required in the ligand-dependent phosphorylation and ubiquitination of RARγ and RXRα. However, ligand-dependent degradation of RARα can take place in the absence of such heterodimerization, and is inhibited by phosphorylation. Kopf et al., J. Biol. Chem. 275:33280 (2000). The RXRα ligand 9-cis retinoic acid has been said to suppress the development of hepatocarcinoma in both experimental and clinical studies. Thus, the binding of an RXR ligand (agonist) both activates RXR and hastens the degradation of the activated RXR. Phosphorylation of RXRα at serine 260 has been found to interfere with this suppression and to inhibit the degradation of RXRα in cultured human HCC (hepatocarcinoma) cells. Adachi et al., Hepatology 35:332-340 (2002).

### SUMMARY OF THE INVENTION

In one embodiment, the present invention is drawn to methods and compositions for the treatment of cancers whose development or proliferation is mediated by β-catenin. Thus herein is disclosed a method of inhibiting the proliferation of a eukaryotic cell whose growth is stimulated by β-catenin-mediated gene transcription, comprising contacting said cell with:

a) a non-endogenous source of RXR nuclear receptor protein, and

b) a therapeutically effective amount of an agonist of said RXR protein.

The inventive methods of this embodiment comprise contacting a target eukaryotic cell or tissue with a non-endogenous source of an RXR nuclear receptor protein. By “non-endogenous source” is meant that the cell is provided with RXR protein in excess of that amount naturally produced by the cell in question. The RXR protein may be provided as part of a fusion protein that stimulates the endocytosis of the RXR moiety, or by other direct means of providing the RXR protein to the cell. Alternatively and preferably, the RXR protein is provided by means of an expressible gene that is internalized within a target cell, for example an expressible RXR gene borne by a viral gene transfer vector. Upon expression of the RXR gene within the target cell, the RXR protein is available to stimulate the ligand-mediated degradation of β-catenin, thus inhibiting the transactivational activity of the β-catenin. Preferably the RXR protein is RXRα. Preferably both the cell and the RXR protein are human.

The non-endogenous source of RXR protein may be provided to the cell by any of a number of means that will be immediately apparent to the person of skill in the art. In one preferred method, the RXR protein is provided indirectly by use of an expression vector carrying an expressible gene encoding the RXR protein. Most preferably, the vector is a viral vector capable of infecting the target tissue.

The use of vectors derived from a virus are capable of delivering a translationally competent RXR gene to the cell along with a strong promoter, and thus permit the transcription of the gene and the production of RXR protein within the nucleus of the cell. Depending upon the nature of the vector and its size, it may also be engineered to contain a foreign, specific RNA polymerase gene, such as, without limitation, the T4 bacteriophage RNA polymerase gene. Since T4 RNA polymerase is quite fastidious in its specificity for its own promoter, and in such a system the transcriptional effects of the vector could be effectively limited to the desired therapeutic effect by placing the translatable nucleic acid region under control of a 5′ T4 promoter sequence. The inclusion of the T4 RNA polymerase gene within the vector, and expression of T4 RNA polymerase within the host cell, would ensure that a large number of copies of the antisense agents would be produced from each vector molecule. Other RNA polymerase genes having strong specificity for particular promoter sequences may also be used, and are known to those of skill in the art.

Viral vectors may be derived from viruses such as, without limitation, adenovirus, adeno-associated virus (e.g., AAV-2), and various retroviruses. Of course, other suitable viral vectors are available or can be envisioned by the person of ordinary skill in the art; the vectors mentioned herein are by way of illustration rather than limitation.

Each prospective vector has its own benefits and deficits. For example, adenovirus infections are common and relatively benign in humans; this virus is one of those responsible for the common cold. The virus contains a double-stranded DNA genome. After deletion of non-essential genes, the virus is able to carry about 8 kilobase pairs of an exogenous double-stranded DNA insert. This amount would be more than adequate to carry the RXR gene as well as necessary regulatory sequences, such as a strong promoter. However, the immunogenicity of adenovirus is relatively high. Additionally, adenovirus does not stably integrate into the host chromosome, and therefore its therapeutic effect is relatively transient; of course, this result may be advantageous when it is desired that the therapeutic effect of the antisense agent be temporary. Certain constructs of adenovirus (and other gene transfer vectors) have been made “replication deficient” in order to control the extent and duration of infection, and to minimize the spread of the recombinant virus.

AAV-2 also commonly infects humans but is not known to cause a disease. The virus is quite small, and therefore it is relatively non-immunogenic. However, the small size also means that there is less room for packaging a therapeutic nucleic acid sequence region and any necessary regulatory sequences or genes such as an RNA polymerase gene. Wild-type AAV-2 stably integrates at a specific site in human chromosome 19, however the gene responsible for this stable integration is deleted in recombinant versions of the viral genome, and this property is therefore lost. Over a period of time recombinant AAV-2 appears to randomly integrate into the host chromosome. Also, optimal gene expression is usually seen after 3-5 weeks when using such vectors.

Retroviruses such as modified Moloney murine leukemia virus have also been used as the raw materials of engineered transfer vectors. The virus gives rise to a minimal immunological response. Retroviral vectors specifically infect dividing cells, and for this reason they appear as attractive candidates as vectors against cancers. Moreover, retroviral vectors stably integrate into the chromosomes of the host cell, providing the potential for long term expression of the passenger nucleic acid, and thus reducing the need for frequent re-introduction of the vector. Construction of retroviral vectors has involved removal of the gag, pol, and env genes from the DNA provirus to make-room for the gene(s) of therapeutic interest, up to about 8 kilobases of inserted nucleic acid. This process makes the vector replication-deficient, and the virus particles are propagated in special “packaging” cell lines that contain the genes missing from the vector. See e.g., The Pharmacological Basis of Therapeutics Ch. 5 (Hardman et al. ed., 9.sup.th ed. 1996), the disclosure of which is hereby incorporated by reference as part of this disclosure.

Other means of providing non-endogenous amounts of an RXR protein to a cell is by stimulation of the expression of the target cell's own RXR gene to produce amounts of RXR in excess of that amount normally found within such cells.

Applicants have discovered that RXR protein stimulates the degradation of β-catenin in a ligand-dependent manner. By “ligand” is meant an agent that is capable of stimulating RXR-mediated gene transcription; that is, an agonist of RXR transcriptional activation. Thus, in the claimed method the target cell is contacted both with the non-endogenous source of RXR protein and with an RXR ligand.

The RXR ligands administered in this invention may be administered systemically or topically, depending on such considerations as the condition to be treated, need for site-specific treatment, quantity of drug to be administered, and numerous other considerations. Thus, in the treatment of cancers, it will generally be preferred to administer the drug directly by injection, or systemically such as by oral or transdermal administration. Any common formulation such as a solution, suspension, gel, ointment, or salve and the like may be used. Preparation of such formulations are well described in the art of pharmaceutical formulations as exemplified, for example, by Remington's Pharmaceutical Science, Edition 17, Mack Publishing Company, Easton, Pa., hereby incorporated by reference herein. If the drug is to be administered systemically, it may be confected as a powder, pill, tablet or the like or as a syrup or elixir suitable for oral administration. For intravenous or intraperitoneal administration, the compound will be prepared as a solution or suspension capable of being administered by injection. In certain cases, it may be useful to formulate these compounds in suppository form or as extended release formulation for deposit under the skin or intramuscular injection.

A “therapeutically effective amount” of the RXR ligand will be that concentration which stimulates RXR-mediated degradation of free β-catenin within the target cell. In certain instances, the compound potentially may be used in prophylactic manner to prevent onset of a particular condition. A useful therapeutic or prophylactic concentration may in certain instances vary with the severity of the condition being treated and the patient's susceptibility to treatment. Accordingly, no single concentration will be uniformly useful, but will require modification depending on the particularities of the disease being treated. Such concentrations can be arrived at through routine experimentation. However, it is anticipated that a formulation containing between 0.01 and 1.0 milligrams per milliliter of formulation will constitute a therapeutically effective concentration for application as an injectable. If administered systemically, an amount between 0.01 and 5 mg per kg of body weight per day would be expected to effect a therapeutic result.

In other embodiments the invention is directed to a method of inhibiting the proliferation of a eukaryotic cell whose growth is stimulated by β-catenin-mediated gene transcription, comprising stimulating the expression of RXR within said cell in the presence of a therapeutic amount of an RXR ligand. The amount of RXR produced within such a stimulated cell is an amount sufficient to cause the degradation of “free” or transcriptionally competent β-catenin when in the presence of a therapeutically effective amount of the RXR ligand.

We have discovered that retinoid X receptor (RXR)-selective compounds such as AGN 4204 induce RXR-mediated degradation of wild type, as well as mutated β-catenin proteins in a dose-dependent manner, resulting in the loss of β-catenin-mediated gene transcriptional activation. We have also found that by elevating RXR levels in cancer cells endogenous β-catenin-mediated gene transactivation is inactivated or inhibited when the elevated RXR levels were accompanied by administration of RXR ligands.

Currently, synthetic RXR ligands are designed and assayed based on interaction between RXRs and their transcriptional co-activators/co-repressors in cell nuclei. Activity of ligands depends on their structure and target protein-protein interaction. The readout of such interaction is generally obtained by the use of a reporter gene containing receptor binding sites linked to a gene coding for an enzyme, whose expression and activity is measurable.

In another embodiment of the present invention, we provide a method of identifying RXR agonists by detecting the RXR-mediated decrease in intracellular β-catenin protein levels and/or β-catenin-LEF/TCF mediated gene transactivation (for example using a LEF/TCF reporter gene) as a function of dose of a test compound.

It is known that RXRs interact with a number of nuclear hormone receptors such as RAR, VDR, TR, LXR, BAR, PXR, NGFIB, and PPAR as well as other cellular proteins such as IGFBP-3. Thus, RXR-selective agonists which influence the intracellular concentration or availability of these other receptors and proteins would be expected to reduce the expression of oncogenic proteins that are pharmacologically regulated by these nuclear receptors or cellular proteins. RXR-selective ligands may also be used to reduce other oncoproteins that contain motifs targeted by the RXR-mediated degradation pathway.

### BRIEF DESCRIPTION OF THE FIGURES

FIG. 1 Inactivation of β-catenin-mediated gene transactivation by RXR-selective retinoid AGN 4204 via RXRα. Cultured cells were transfected with 100 ng of reporter gene Topflash® together with expression vectors indicated under each graph. After transfection, cells were treated with vehicle or 0.1 μM AGN 4204 for 17 hours. (A) Data from HEK293 cells co-transfected with wild type or mutant β-catenin (200 ng) and RXRα (20 ng). (B) CAT, a stable cell line that overexpresses β-catenin (left panel). RBC, a stable cell line that overexpresses both RXRα and β-catenin. (C) SW480, a colorectal cancer cell line, was transfected with 100 ng of Topflash®. The amount of RXRα cotransfected was indicated below the X axis. Reporter activity on the Y axis is expressed as either Luciferase Unit or percentage of activity in cells transfected with Topflash® alone.

FIG. 2 RXR-dependent degradation of β-catenin protein by RXR ligand AGN 4204. (A) Western blotting analysis of HEK293 cells transfected with a combination of expression vectors for LacZ (2 μg), V5-tagged β-catenin(4 μg), and Flag-tagged RXRα (2 μg) as indicated below the gels. Cells were treated with vehicle “−” or 0.1 μM AGN 4204 “+” for 6 or 15 hours as indicated above the gels. β-galactosidase was detected by a mouse monoclonal antibody. β-catenin protein was analyzed using an HRP-conjugated mouse monoclonal antibody against the V5 tag. RXRα protein was measured using an HRP-conjugated mouse monoclonal antibody against the Flag tag. (B) Western blotting analysis of stable cell line RBC that overexpresses both β-catenin and RXRα. Cells were treated with vehicle or 0.1 μM AGN 4204 for duration indicated above the gels. (C) Western blotting analysis of stable cell line CAT that overexpresses only β-catenin. The left panel shows CAT cells transfected with the parental empty expression vector. The right panel shows CAT cells transfected with 2 μg of expression vectors for RXRα. Cells were treated with vehicle or 0.1 μM AGN 4204 for 17 hours. Endogenous and overexpressed β-catenin were detected using a rabbit polyclonal antibody as indicated by “Total” whereas overexpressed β-catenin was analyzed using a mouse monoclonal antibody against the V5 tag. Endogenous and overexpressed RXRα were detected by a rabbit polyclonal antibody against RXRα, whereas overexpressed RXRα was determined by an HRP-conjugated mouse monoclonal antibody against the Flag tag. (D) Western blotting analysis of HEK293 cells transfected with a fixed amount of β-catenin (4 μg) plus an increasing amount of RXRα as indicated at the top. Cells were treated with vehicle or 0.1 μM AGN 4204 for 17 hours. (E) Western blotting analysis of HEK293, CV-1, and Hela cells transfected with 2 μg of RXRα and 4 μg of wild type or mutant β-catenin. α. Cells were treated with vehicle or 0.1 μM AGN 4204 for 17 hours. (F) Northern blotting analysis of RBC cells using β-catenin as a probe. Cells were treated for 17 hours with vehicle or AGN 4204 at concentrations indicated above the gel.

FIG. 3 Time course of degradation of the mutant β-catenin by AGN 4204 in stable cell line RmBC. RmBC overexpresses both RXRα and the β-catenin mutant lacking the N-terminal first 50 amino acids (ΔNβ-catenin). Cells were treated with vehicle or 0.1 μM AGN 4204 for duration indicated above the gels. The levels of ΔNβ-catenin and RXRα were determined by Western blotting.

FIG. 4 Receptor and ligand specificity in degradation of β-catenin by retinoid receptors. HEK293 cells transfected with expression vectors for β-catenin (4 μg) and RXRs (2 μg) or RARs (2 μg) and treated with ligands at concentrations indicated at the top of each gel. Protein levels were detected by Western blotting as shown in this figure. β-catenin and RARs were detected by HRP-conjugated antibodies against their V5 tag. RXRs were detected by HRP-conjugated antibodies against their Flag tag. (A) shows the high potency of AGN 4204 in inducing β-catenin degradation and that this degradation activity can be inhibited by RXR antagonists 195393. (B) shows that several RXR-specific agonists (Lanes 2-7) are able to induce this degradation whereas RAR agonist TTNPB, RXR antagonist AGN195393, and RAR antagonist AGN194310 have no effects. (C) shows that RXRγ also has the ability to induce the degradation of β-catenin. (D) shows that RARs do not have significant activity in degradation of β-catenin. (E) shows that RXR and its ligand AGN 4204 are the key determinants in induction of degradation of β-catenin and RXR dimerization partners RARs. In this experiment, the amount of expression vectors for RXRα and RARs used in transfection was 1 μg.

FIG. 5 Integrity of RXRα is required for its activity in induction of β-catenin degradation by AGN 4204. (A) Diagram shows functional domains that were deleted in RXRα mutants. AF-1, transactivation function-1; DNA, DNA binding domain; Ligand, ligand binding domain; Dimer, dimerization domain; AF-2, activation function-2 domain. Solid bars indicate regions retained in the mutants. (B) Western blotting analysis of HEK293 cells transfected with β-catenin (2 μg) and RXRα mutants (4 μg). Cells were treated with vehicle or 0.1 μM AGN 4204 for 17 hours. (C) The dose-dependent effects of AGN 4204 on luciferase reporter activity in CV1 cells transfected with CRBPII-TK-Luc and RXRα deletion mutants.

FIG. 6 Interaction of β-catenin with RXRα. HEK293 cells were transfected with a combination of 8 μg of ΔNβ-catenin (C) and 4 μg of RXRα. Cells were treated with vehicle or 1 μM AGN 4204 for 15 min. before the crosslinking reaction. Cell lysates were subjected to immunoprecipitation using antibodies against the Flag tag in RXRα. Crosslinked molecules in immunoprecipitates were dissociated by reduction with β-mercaptoethanol before Western blotting analysis using HRP-conjugated antibodies against the v5 tag in ΔNβ-catenin (Top panel). Lower panel shows the direct Western blotting analysis of cell lysates reduced with β-mercaptoethanol. IP, immunoprecipitation; M2, antibody against the Flag tag in RXRα; IB, immunoblotting (Western blotting); V5-HRP, HRP-conjugated antibody against the V5 tag in ΔNβ-catenin.

### DETAILED DESCRIPTION OF THE INVENTION

In a major embodiment the present invention is directed to methods for the treatment of cancers whose development and/or progression is related to the aberrant transcription of genes whose expression is positively regulated by β-catenin. Such cancers particularly include, without limitation, colon cancer.

β-catenin is a key regulator in cell-cell adhesion, cell differentiation, proliferation and motility during embryo development. When complexed with the APC protein, excess β catenin is targeted for elimination by proteasomes through the ubiquitin mediated degradation pathway. Dysregulation of or dissociation from the APC pathway leads to the free form of β-catenin being localized to the nucleus, and activation of gene transcription through the LEF/TCF pathway. In adults, such dysregulation, which can be caused by mutations in β-catenin or APC, is believed to be implicated in colorectal cancer and many other forms of cancer. Mutations of the APC protein occur in more than 70% of all colorectal cancers. Chung, D., Gastroenterology 119:854-865 (2000).

Retinoids also play many important roles in cell differentiation, proliferation and apoptosis in embryo development and adult homeostasis. Retinoids are well known as exerting a direct action on gene transcription; by binding to their receptors they induce a change in receptor conformation and therefore cause the bound receptor to bind to cognate motifs in the regulatory regions of target genes. Binding of retinoid or other receptor agonists to their cognate substrate, retinoid X receptor-α (RXRα), causes the destruction of the RXR protein. However, the biological consequences of this event have not been well understood.

We have found that RXR-selective ligands induce the degradation of α-catenin proteins, resulting in the loss of the α-catenin:LEF/TGF-mediated gene activation. This process requires the presence of RXR, which itself is subjected to degradation in a ligand-dependent manner. Binding of ligands to RXR also causes degradation of the RAR portions of RXR:RAR heterodimers, all three subtypes of the retinoic acid receptor (RAR) family (RARα, RARβ and RARγ) are so degraded.

We have shown that deletion of the GSK3β-targeted N-terminal peptide of β-catenin does not impair its degradation as well as reduction of the β-catenin reporter gene activity by RXR ligands, indicating that RXR-mediated degradation pathway is independent of GSK3β. We also found that β-catenin interacts with RXRα in ligand-independent manner. Elevating RXR levels in colorectal cancer cells led to inactivation of endogenous β-catenin-mediated gene transactivation in response to RXR ligands.

### Experimental Procedure

The β-catenin reporter plasmid Topflash® was purchased from Upstate Biotechnology. The β-catenin expression vector, Gene Storm® clone H-X87838M in pcDNA3.1/GS, was purchased from Invitrogen Corporation. A β-catenin mutant containing an N-terminal deletion of 50 amino acids (termed “ΔNβ-catenin” herein), was made by PCR amplification from DNA encoding template wild type β-catenin using the following pair of primers (all nucleotide sequences are shown in the orientation, from right to left of 5′ to 3′):

SEQ ID NO: 1

AGG GAT CCA ACC ATG AAT CCT GAG GAA GAG,
and

SEQ ID NO: 2

AGTCTAGATTACAGGTCAGTATCAAACCAG

The resulting DNA fragment (whose N terminus lacked the first 50 amino acids of wild-type β-catenin) was cloned in expression vector pcDNA3.1+ (Invitrogen Corp.) between the BamHl and the Xbal restriction endonuclease sites—the sequence of the fragment was confirmed by DNA sequencing. Finally, the fragment containing the deletion was released by digestion with endonucleases Mnul and Xhol and used to replace the 5′ terminus of wild type β-catenin in pcDNA3.1/GS (pGS-β-catenin).

Human RXRα cDNA in a human keratinocyte cDNA library (Nagpal et al., 1999, J. Biol. Chem. 274:22563-22568) was identified in a yeast two-hybrid system using RARγ as a bait. The RXRα coding region was amplified from this clone by PCR using the following primers:

SEQ ID NO: 3

AG GAA TTC ATG GAC ACC AAA CAT TTC CTG CCG,
and

SEQ ID NO: 4

AG CTG CAG CTA AGT CAT TTG GTG CGG CGG CTC

The resulting fragment was subcloned into pEGFP-N2 (Clontech) between the EcoRI and PstI sites in the cloning cluster and then released by EcoRI and KpnI digestion. The released RXRα coding region was then cloned into a modified pCMV-Flag vector (Sigma) containing the Flag epitope

DYKDDDDK. SEQ ID NO: 5

The RXRα deletion mutants were constructed by PCR amplification of hRXRα cDNA using primer pairs specific for different regions (see Table 1). The resulting PCR fragments were cut by EcoRl and Kpnl and cloned into the pCMV-Flag vector. For construction of RXRαΔC and RXRαΔCD, the EcoRl fragment obtained from PCR amplification of the A/B region of RXRα was inserted into RXRαDE and RXRαE respectively at the EcoRl site in front of the DE and E regions of RXRα. For RXRαΔD, the EcoRl fragment from the amplification of the ABC region of RXRα was inserted into RXRαE.

Human RXRγ was cloned by PCR from a brain cDNA library (Clontech) using the primers indicated in Table 1. The amplified fragment was cloned into pCMV-Flag between the EcoRl and Kpnl sites.

Expression vectors for all three subtypes of RAR were described previously (Klein E. S. et al. 2000, J. Biol. Chem. 275:19401-19408). In these vectors, the C-terminus of RARs is tagged with a V5 epitope.

TABLE 1

Nomenclature and PCR oligonucleotide primers for
hRXRα and hRXRγ mutants.

| | |
|---|---|
| Mutants | PCR Primers |

| | |
|---|---|
| RXRα CDE | SEQ ID NO: 6 |
| | AGGAATTCTGCGCCATCTGCGGGGACCGC |
| | SEQ ID NO: 7 |
| | AGGGTACCCTAAGTCATTTGGTGCGGCGCCTCC |
| RXRα DE | SEQ ID NO: 8 |
| | AGGAATTCAAGCGGGAAGCCGTGCAGGAGGAGCGG |
| | SEQ ID NO: 9 |
| | AGGGTACCCTAAGTCATTTGGTGCGGCGCCTCC |
| RXRα E | SEQ ID NO: 10 |
| | AGGAATTCTCGCCGAACGACCCTGTCACC |
| | SEQ ID NO: 11 |
| | AGGGTACCCTAAGTCATTTGGTGCGGCGCCTCC |
| RXRα ΔC | SEQ ID NO: 12 |
| | AGGAATTCATGGACACCAAACATTTCCTGCCG |
| RXRα ΔCD | SEQ ID NO: 13 |
| | AGGAATTCGATGTGCTTGGTGAAGGAAGCC |
| RXRα ΔD | SEQ ID NO: 14 |
| | AGGAATTCGATGCCCATGGCCAGGCACTTC |
| RXRα ΔAAF2 | SEQ ID NO: 15 |
| | AGGAATTCATGGACACCAAACATTTCCTGCCG |
| | SEQ ID NO: 16 |
| | GGGTACCCTAGATGAGCTTGAAGAAGAAGAG |
| RXRα | SEQ ID NO: 17 |
| | AGGAATTCTGCGCCATCTGCGGGGACCGC |
| CDEΔAF2 | SEQ ID NO: 18 |
| | AGGGTACCCTAGATGAGCTTGAAGAAGAAGAG |
| RXRα DEΔAF2 | SEQ ID NO: 19 |
| | AGGAATTCAAGCGGGAAGCCGTGCAGGAGGAGCGG |
| | SEQ ID NO: 20 |
| | AGGGTACCCTAGATGAGCTTGAAGAAGAAGAG |
| RXRα EΔAF2 | SEQ ID NO: 21 |
| | AGGAATTCTCGCCGAACGACCCTGTCACC |
| | SEQ ID NO: 22 |
| | AGGGTACCCTAGATGAGCTTGAAGAAGAAGAG |
| RXRγ WT | SEQ ID NO: 23 |
| | AGGAATTCATGTATGGAAATTATTCTCACTTC |
| | SEQ ID NO: 24 |
| | AGGGTACCTCAGGTGATCTGCAGCGGGGTCTCC |

### Antibodies

Unconjugated and horseradish peroxidase (HRP)-conjugated mouse monoclonal antibodies against the Flag tag (M2 and HRP-M2) were purchased from Sigma, Mo. Unconjugated and horseradish peroxidase(HRP)-conjugated mouse monoclonal antibodies against the V5 tag (V5 and HRP-V5) were purchased from Invitrogen Corp., CA. Rabbit polyclonal antibodies against the N-terminus of RXRα (D20) or the C-terminus of β-catenin (H102) were from Santa Cruz Biotechnology Inc., CA.

### Cell Lines

HEK293, Hela, CV1, and SW480 cells were purchased from ATCC and grown in Dulbecco's modified Eagle's medium (DMEM) supplemented with 10% fetal bovine serum, 100 U of penicillin per ml and 10 μg of streptomycin per ml at 37° C. in 5% CO₂.

To generate a cell line (termed “CAT”) that stably overexpresses β-catenin, pGS-β-catenin was transfected into HEK293 cells using Lipofectamine®. Twenty-four hours after the transfection cells were subjected to selection in the presence of zeocin (Invitrogen) at a concentration of 400-500 μg/ml. The selection medium was changed every 3 days and individual zeocine-resistant clones were isolated. Western blotting analysis with the V5 antibody identified clones stably expressing β-catenin.

To produce RBC and RmBC cell lines that stably overexpress RXRα with wild type β-catenin or mutant ΔNβ-catenin, pGS-β-catenin and pGS-ΔNβ-catenin were transfected into cell line F19 that overexpresses only RXRα. These cell lines were constructed in a manner similar to that described for the CAT cell line.

### Reporter Gene Assays

For transfection of the HEK293 cell line, cells were seeded at a density of 50,000 cells per well in 24-well plates coated with poly-D-Lysine (Becton Dickinson). After 24 hours, the luciferase-based reporter plasmids and expression vectors were cotransfected into cells using Fugene according to the manufacturer's instruction (Roche Applied Science). To monitor the efficiency of transfection, either 15 ng of phRG-TK Renilla or 100 ng of CMX-LacZ DNA were cotransfected. Five hours later, transfection media were replaced with fresh media containing 10% charcoal-treated FBS(fetal bovine serum) with dimethylsulfoxide (DMSO) (≦0.1%) or retinoids prepared in DMSO, and the cells incubated for another 24 hours before harvest. Harvested cells were lysed, and luciferase activity was measured as previously described (de Wet, J. R., Wood, K. V., DeLuca, M., Helinski, D. R., Subramani, S. (1987) Mol. Cell. Biol. 7, 725-737). In cells transfected with the LacZ control gene, β-galactosidase activity was measured by standard colorimetric assays. Renilla reniformis GFP activity was determined using the Dual-Luciferase Reporter 100 Assay System (Promega Corporation, 2800 Woods Hollow Road, Madison Wis. USA 53711). The experimental reporter gene activity was normalized against either β-galactosidase or Renilla GFP activity. Values represent the mean +/− SEM of quadruplicate determinations.

Analysis of ligand regulation of RXRα and its mutants in transactivation was performed as follows. 3.5×10³ CV-1 cells were seeded in each well of a 96-well opaque plate (Falcon). The cells were transiently transfected via Lipofectamine® with the reporter plasmid CRBPII-tk-luc together with 0.04 ug of RXRα WT, and the mutants RXRαCDE, RXRαDE, RXRαE, RXRαΔC, RXRαΔD, RXRαΔCD, RXRαΔAF2. After 5 hours of introduction of DNA, cells were fed with DMEM (Dulbecco's modified Eagle's medium) containing 20% charcoal treated FBS. Cells were treated with retinoids for 18 hours and lysed. Luciferase activity was measured as previously described (de Wet, J. R., Wood, K. V., DeLuca, M., Helinski, D. R., Subramani, S. (1987) Mol. Cell. Biol. 7, 725-737). Luciferase values represent the mean +/− SEM of quadruplicate determinations.

### Protein Expression and Western Blotting Analysis

HEK293 cells were seeded at a density of 2×10 per dish were seeded into 100-mm dishes and cultured overnight in DMEM containing 10% FBS. Cells were transfected with 1-4 μg of various cDNA or parental expression vectors (8 μg of DNA in total) using Lipofectamine®. Five hours after transfection, cells were fed with fresh culture media DMEM containing 10% charcoal-treated FBS with vehicle DMSO or retinoids prepared in DMSO. After a given time of treatment, cells were harvested and lysed in a buffer containing 1% Nonidet® P-40, 30 mM Tris-HCl (pH7.4), 0.5 mM EDTA (pH 8.0), 150 mM NaCl, 10% glycerol, 1 mM sodium orthovanadate, 40 mM NaF, 0.5 mM phenylmethylsulfonyl fluoride (PMSF) and a cocktail of protease inhibitors (Merck).

Total cell lysates were homogenized by passing through a QIAshredder® (Qiagen) and cleared from insoluble materials by centriftigation at 12,000 g. Protein concentration was determined using a Bradford total protein assay kit (Bio-Rad). Total cell lysates were electrophoresed using a 4-12% SDS-PAGE gradient gel and transferred to either nitrocellulose or PVDF filter membranes. The membranes were blocked with 10% non-fat milk reconstituted from dairy milk powder in phosphate-buffered saline (PBS) containing 0.1% Tween®-20 (PBST). The membranes were incubated with primary antibodies at room temperature for 2 hours or at 4° C. for overnight. After the removal of unbound antibodies, membranes were incubated with horseradish peroxidase-conjugated secondary antibodies for one hour at room temperature and washed five times with PBST. The antibody-associated protein bands were revealed by the chemiluminescence technique using the ECL Plus system (Amersham).

### In Vivo Protein Crosslinking

HEK293 cells were cultured to about 80% confluence in 150-mm Biocoat poly-D-Lysine plates (VWR Cat#354550), and then transfected with expression vectors for ΔN-β-catenin, and RXRα using Lipofectamine® Transfection Reagent (Invitrogen). Cells were then cultured in high glucose DMEM medium containing 10% activated charcoal-extracted fetal bovine serum. The next morning, cells were treated with 0.1% DMSO or 1 uM AGN 4204 in DMSO for 20 minutes, then the culture medium was removed, and the cells were crosslinked with 1 mM of the reversible crosslinking reagent DSP [dithiobis(succinimidylpropionate)] (Pierce, Cat#22585) in PBS (phosphate-buffered saline) for 15 min.

The structure of AGN 4204 is as follows:

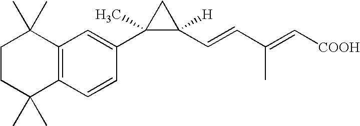

The reaction was quenched by adding Tris buffer at pH7.5 to a final concentration of 20 mM. Cells were lysed in cold RIPA buffer (150 mM NaCl, 1% Nonidet P-40, 0.5% Na-deoxycholate, 0.1% SDS in PBS buffer) containing a protease inhibitor cocktail (Sigma, Cat#P8340), and homogenized using a QIAshredder (Qiagen). Then, 1.5 mg of the extracts were used per immunoprecipitation reaction. Specific antibodies (mouse anti-V5, Invitrogen; or mouse anti-M2, Sigma) and protein G-agarose beads were added and followed by overnight incubation with constant shaking at 4° C. After washing with ice-cold RIPA buffer, immunoprecipitated materials were dissolved in SDS-PAGE loading dye containing β-mercaptoethanol by heating at 100° C. for 5 min. This procedure frees the DSP-crosslinked molecules pulled-down by Protein G-beads. Supernatants were resolved on 4-12% SDS-polyacrylamide gels followed by Western blotting.

### EXAMPLE 1

### RXR-Dependent Reduction of β-Catenin Reporter Gene Activity

To investigate whether RXR and its ligands affect β-catenin-mediated gene transactivation, the APC-positive human HEK293 cells were transfected with the reporter gene Topflash®. This reporter gene contains multiple copies of responsive elements for the DNA-binding transcription activators, TCF/LEF, upstream of the basic luciferase reporter gene construct tk-Luc. The tk-Luc portion of he reporter plasmid consists of a minimal gene promoter from HSV tk gene and a coding sequence for luciferase. Transactivation of this reporter by TCF/LEF can be affected by endogenous as well as overexpressed β-catenin, which interacts with TCF/LEF as a co-activator.

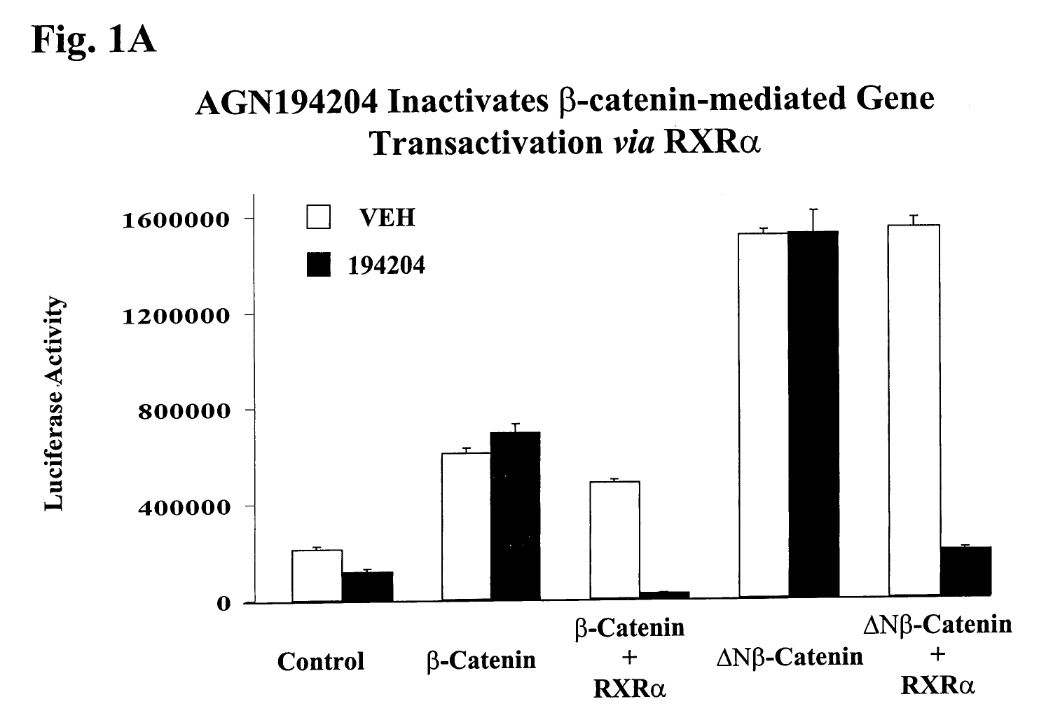

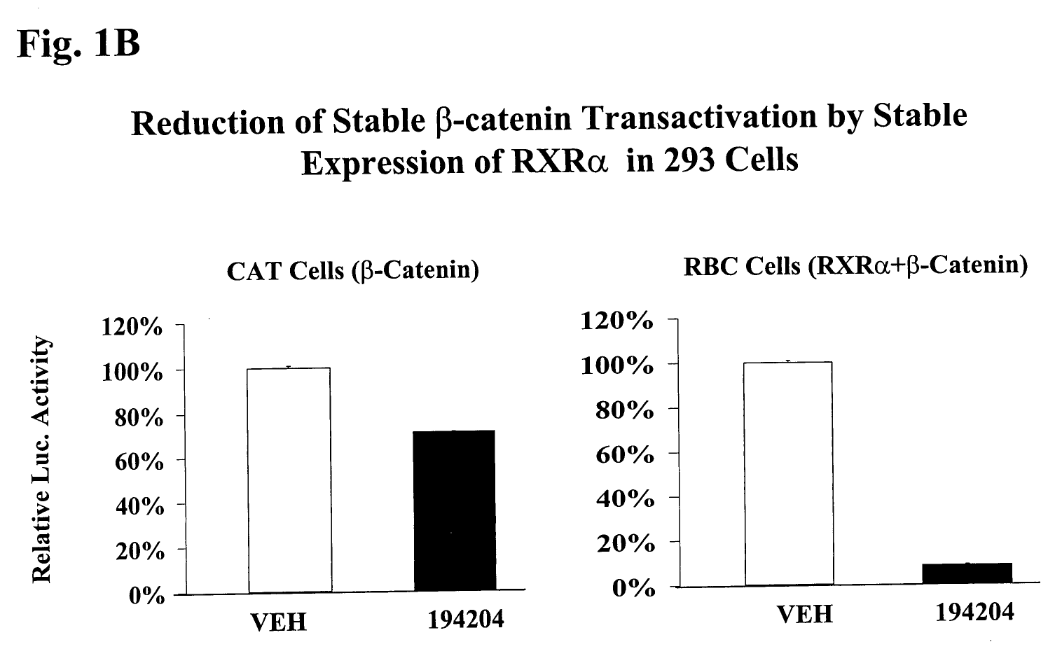

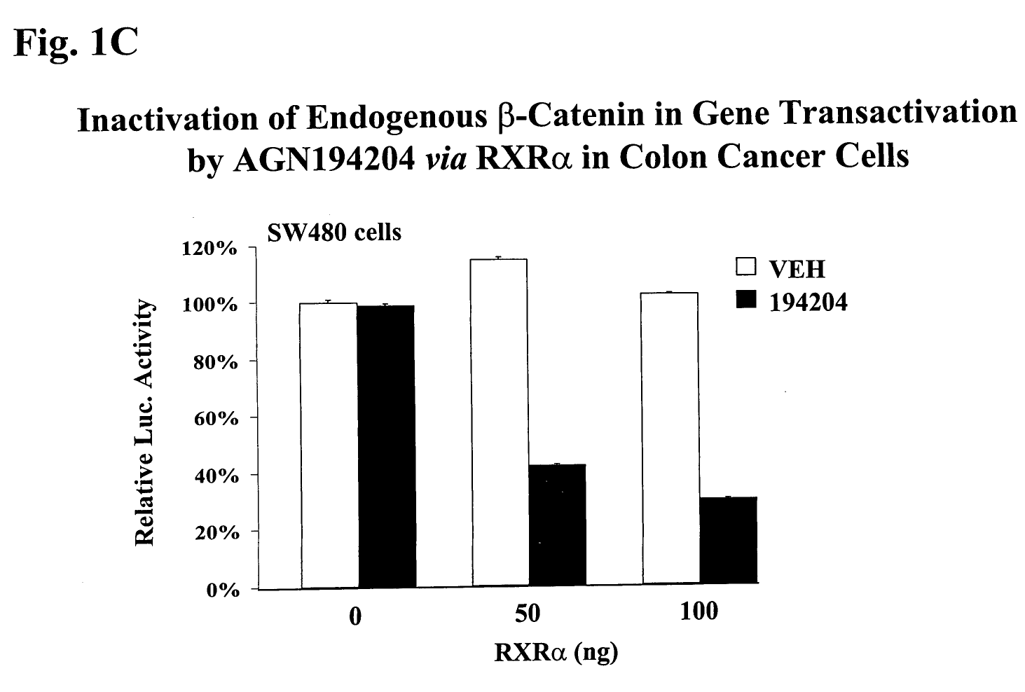

As shown in FIG. 1A, the endogenous β-catenin produced a significant reporter signal, as shown by luciferase activity. This endogenous transcription potentiating activity was moderately reduced by treating cells with the RXR-selective retinoid AGN 4204. Co-transfection of an expression vector for β-catenin alone significantly increased reporter activity. Cotransfection of the HEK293 cells with both β-catenin and RXRα and incubation with AGN 4204 resulted in a 90% decrease in reporter activity, to levels lower than seen in the absence of exogenous β-catenin. The AGN 4204-induced decrease in the reporter activity was also observed in a stable HEK293 cell line, RBC, which stably expresses both β-catenin and RXRα (FIG. 1B). However, in the precursor cell line, CAT, which only overexpresses β-catenin, this RXR ligand only caused a minimal decrease (30%) in the reporter activity. In the APC-negative colorectal cancer cell line SW480, high levels of β-catenin-driven gene transcription were observed with the Topflash® reporter gene (FIG. 1C), consistent with the previously reported observation (Ref). When RXRα was cotransfected with this reporter, a decrease in reporter gene activity by AGN 4204 was observed in the SW480 line (FIG. 1C), while low levels of endogenous RXRs (data not shown) were not sufficient to produce an effect. All these reporter gene data indicate that the RXR selective-retinoid, AGN 4204 is able to reduce gene transactivation by β-catenin via RXRα.

### EXAMPLE 2

### RXR-Dependent Reduction of β-Catenin Protein

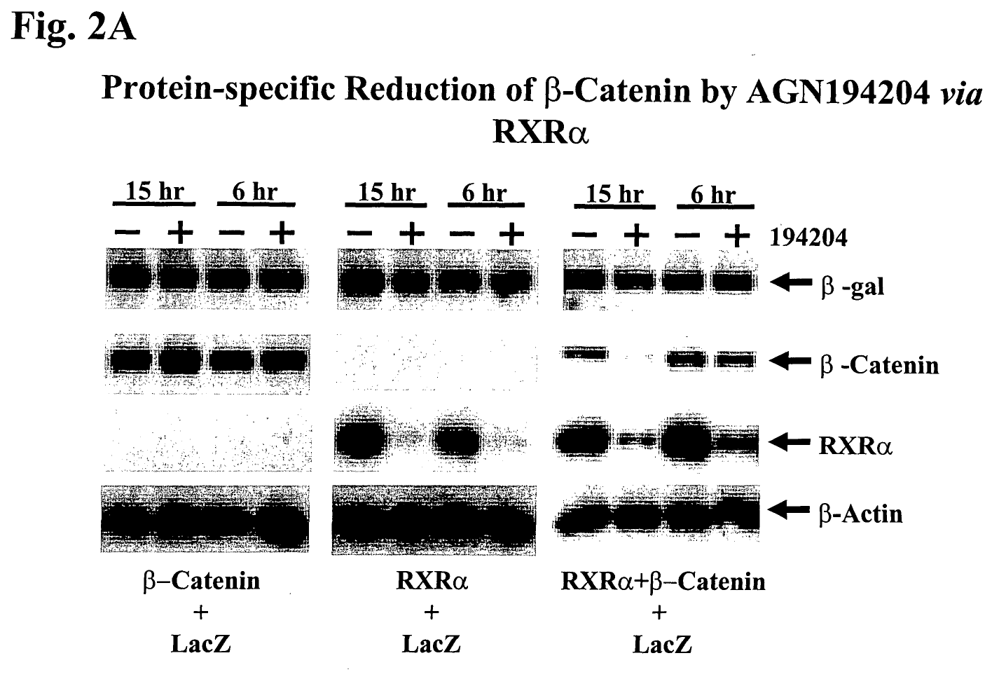

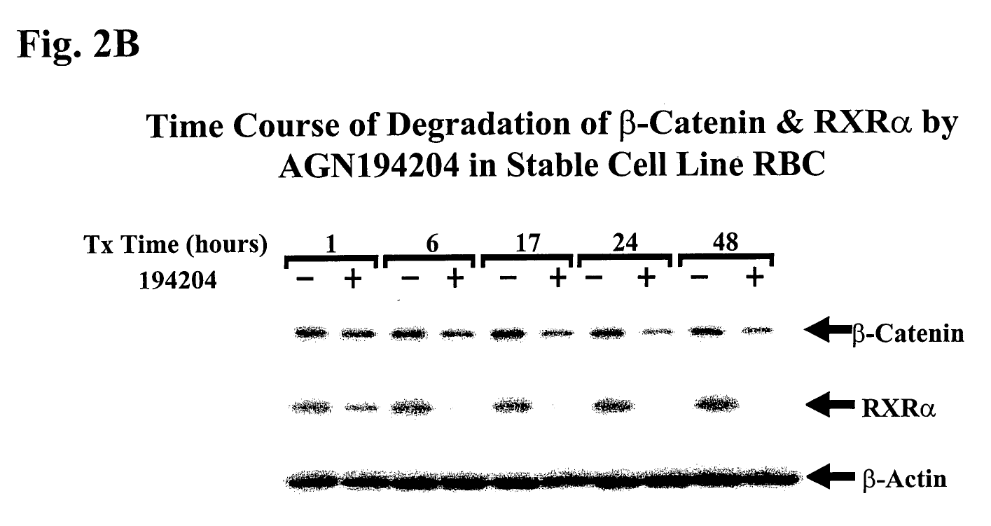

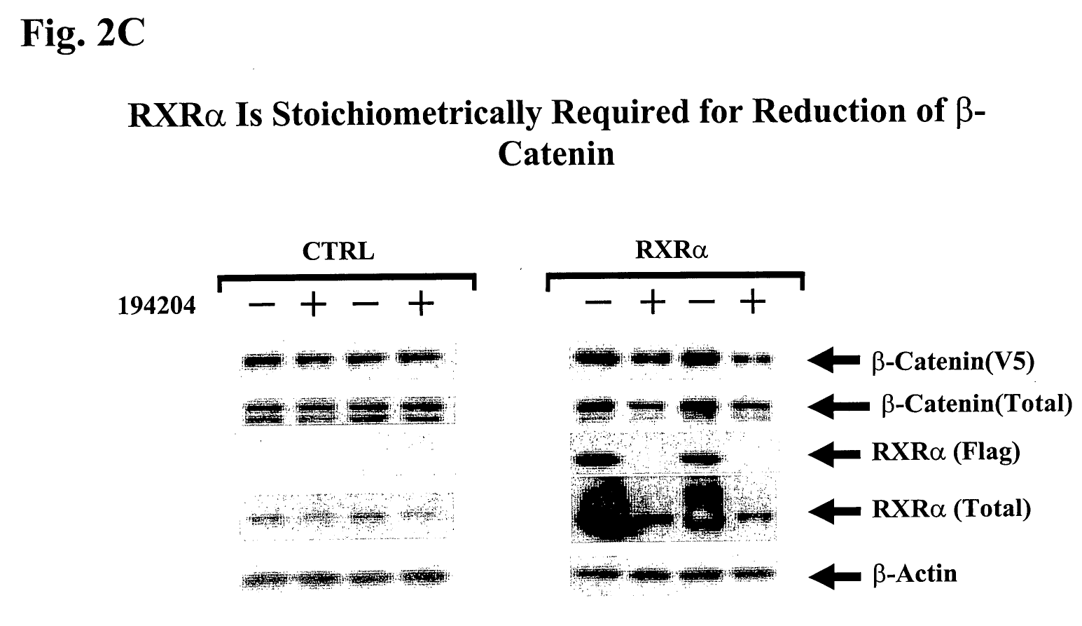

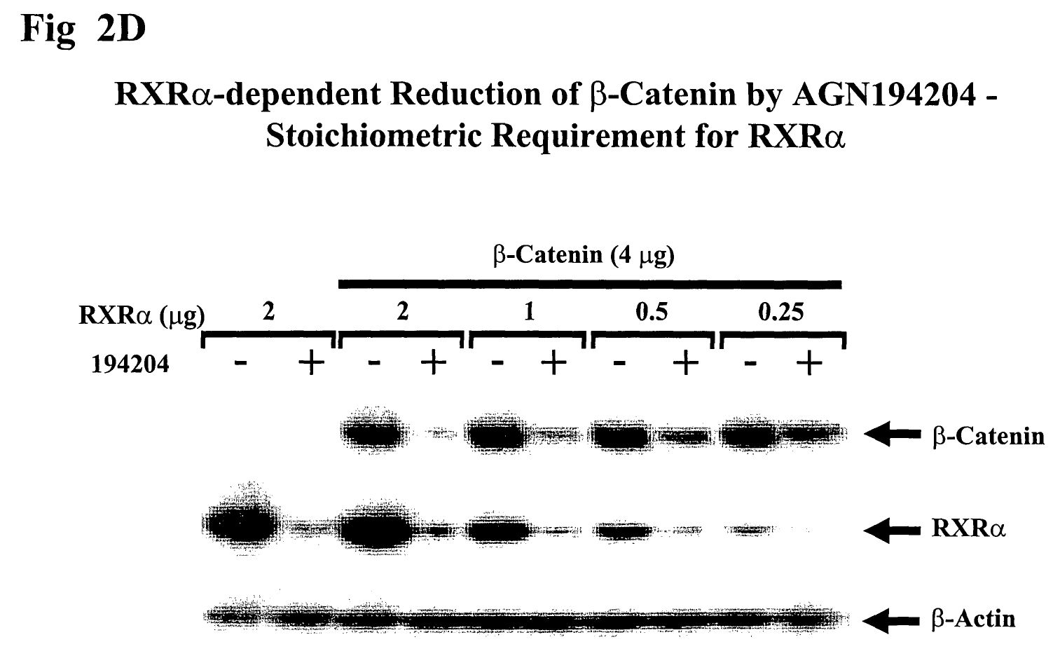

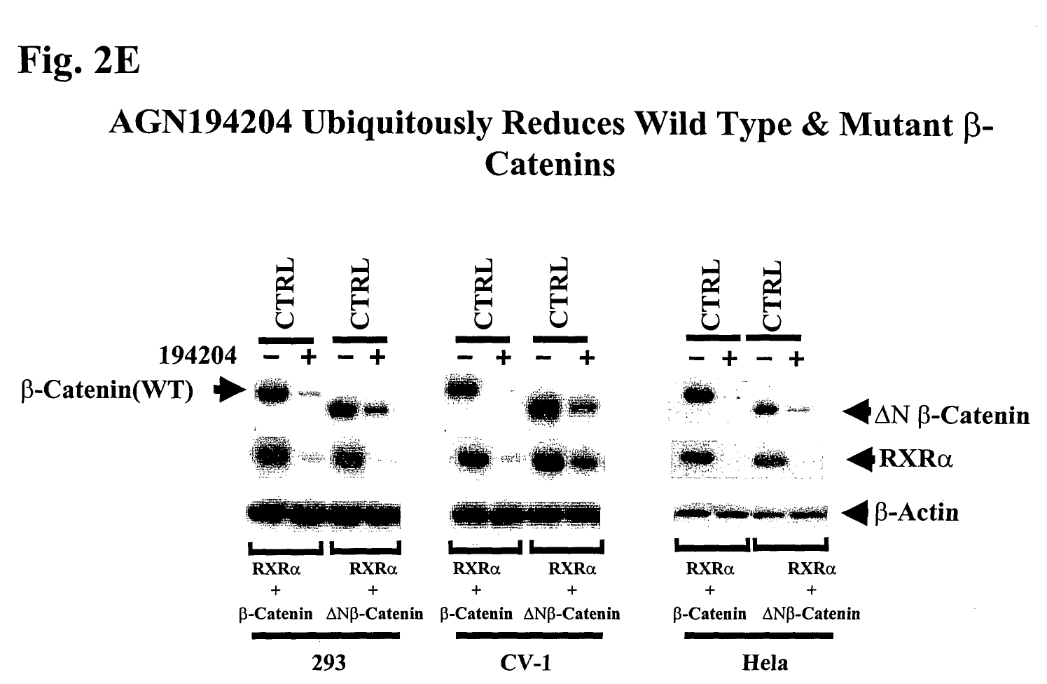

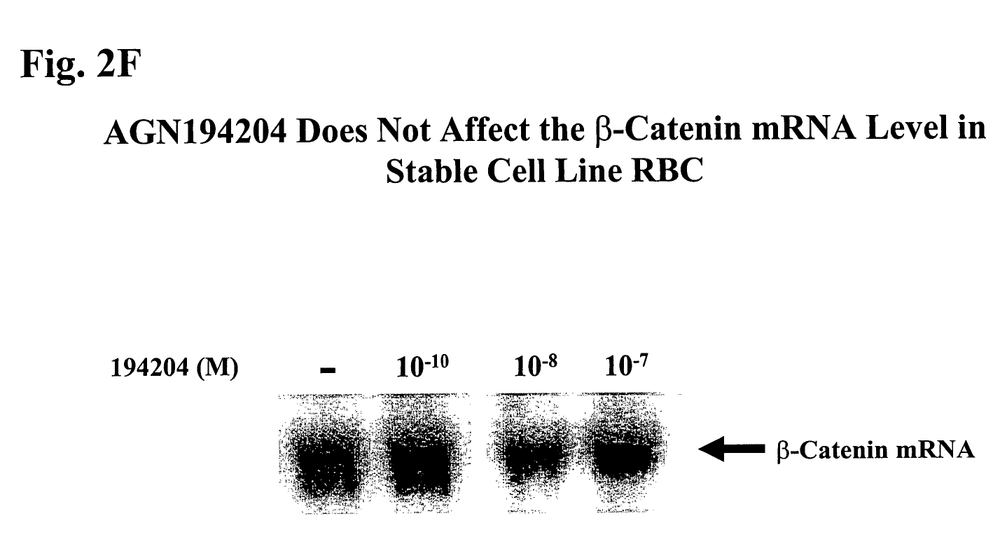

To know whether the RXR-mediated reduction of the reporter gene activity was caused by a decrease in the amount of the β-catenin protein, Western blotting analysis was carried out on lysates of HEK293 cells transfected with expression vectors for β-catenin and/or RXRα. As a control, an expression vector for the bacterial LacZ gene was cotransfected. As shown in FIG. 2A, when β-catenin alone was overexpressed, the RXR ligand AGN 4204 had no significant effects on the intracellular β-catenin protein levels. However, co-overexpression of β-catenin and RXR led to a reduction in the amounts of β-catenin protein. In contrast, expression of the β-galactosidase protein from the Lac Z gene was not significantly affected.

Similar results were obtained with a stable HEK293 cell line RBC expressing β-catenin together with RXRα (FIG. 2B). The RXR ligand also caused a reduction inn the amount of RXRα, independent of β-catenin (FIG. 2A). Degradation of RXRα was detected as early as 1 hour after treatment with AGN 4204 in the RBC cells (FIG. 2B). However, in the same cells, the beginning of β-catenin degradation was observed after 6 hours of treatment. As shown in FIG. 2C, in the CAT cells, which overexpress β-catenin alone, low amounts of endogenous RXRα did not cause a significant decrease of β-catenin in the presence of AGN 4204 for 17 hours, although a decrease of the endogenous RXRα was observed. However, when the CAT cells were transfected with expression vectors for RXRα, β-catenin was significantly reduced in response to administration of AGN 4204 (FIG. 2D). In transiently transfected HEK293 cells, increasing amounts of the RXRα expression vector led to a proportional decrease in β-catenin in response to AGN 4204. Ligand-dependent reduction of β-catenin by RXRα is not restricted to the HEK293 cells. As shown in FIG. 2E, The RXR ligand effects on β-catenin was also observed in other cell types such as CV-1 and Hela cells, indicating the ubiquitous nature of this molecular action.

To exclude the possibility that reduction of β-catenin levels occur at the mRNA level, total RNA was isolated from the RBC cells treated overnight with AGN 4204. As shown in FIG. 2F, AGN 4204 administration had no effects on the β-catenin mRNA level. Taken together, these results indicate that RXR ligand AGN 4204 causes a reduction in β-catenin protein through an RXR-mediated pathway.

### EXAMPLE 3

### The GSK3β-Targeted Sequence in β-Catenin is not Required for RXR Ligand-Induced Reduction of β-Catenin

The amount of free intracellular β-catenin is normally regulated by the APC-mediated degradation pathway. After binding to APC, phosphorylation of serine residues by GSK3β in the first 50 amino acids of β-catenin leads to its ubiquitination and proteasome-mediated degradation. Mutations of these β-catenin serine residues have been found in cancer patients and have been shown to coincide with an increase in β-catenin transactivation activity in cultured cell systems. To know whether this GSK3β-targeted sequence is required for the RXR ligand effect described above, a β-catenin mutant with deletion of the first 50 amino terminal residues, termed ΔNβ-catenin, was constructed.

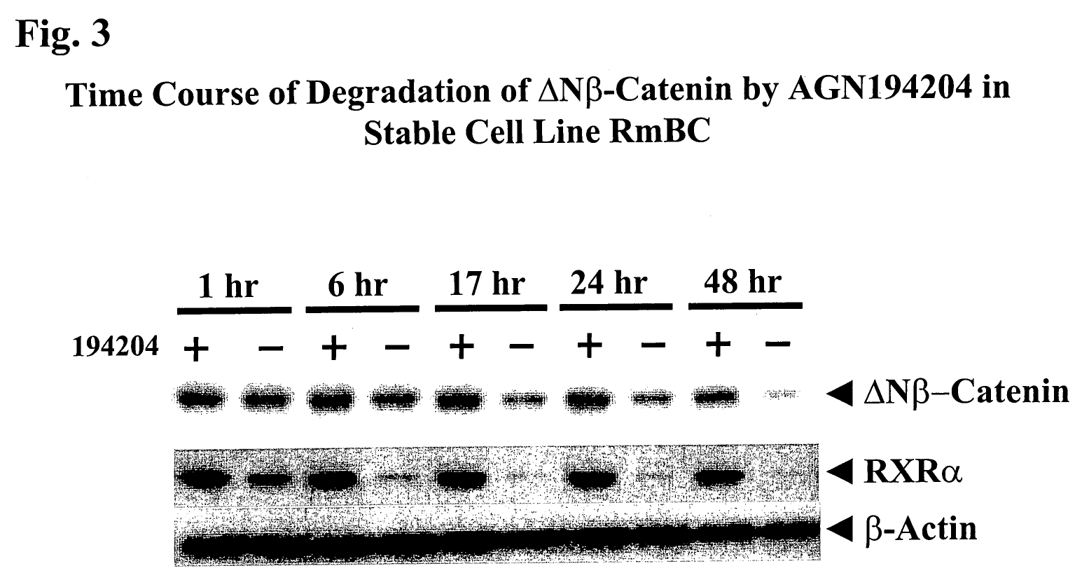

As expected, this mutant displayed higher activity than the wild type β-catenin in transactivating reporter gene Topflash® in HEK293 cells (FIG. 1A). Like its wild type counterpart, the mutant activity is significantly impaired by AGN 4204 in the presence of RXRα. At the protein level, RXR-mediated degradation of αNβ-catenin was observed in transiently transfected HEK293 cells (FIG. 2E) and a stable cell line RmBC overexpressing both ΔNβ-catenin and RXRα (FIG. 3). Like its wild type counterpart, ΔNβ-catenin was found susceptible to RXR-mediated degradation in CV1 and Hela cells (FIG. 2E). Our observations thus indicate that the GSK3β phosphorylation sites in β-catenin are not required for RXR-mediated degradation.

### EXAMPLE 4

### Ligand and Receptor Specificity in the Retinoid-Induced Reduction of β-Catenin

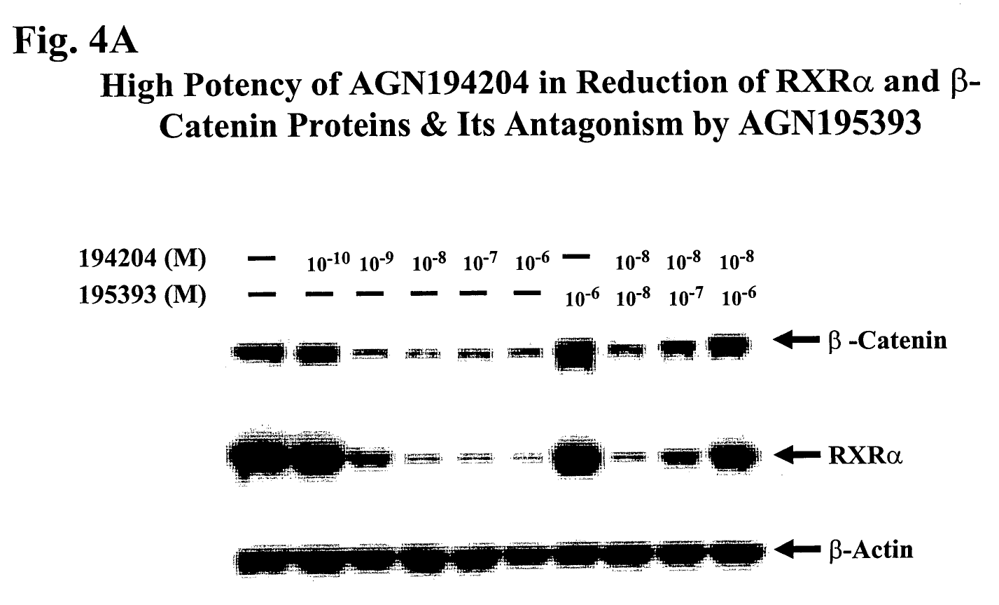

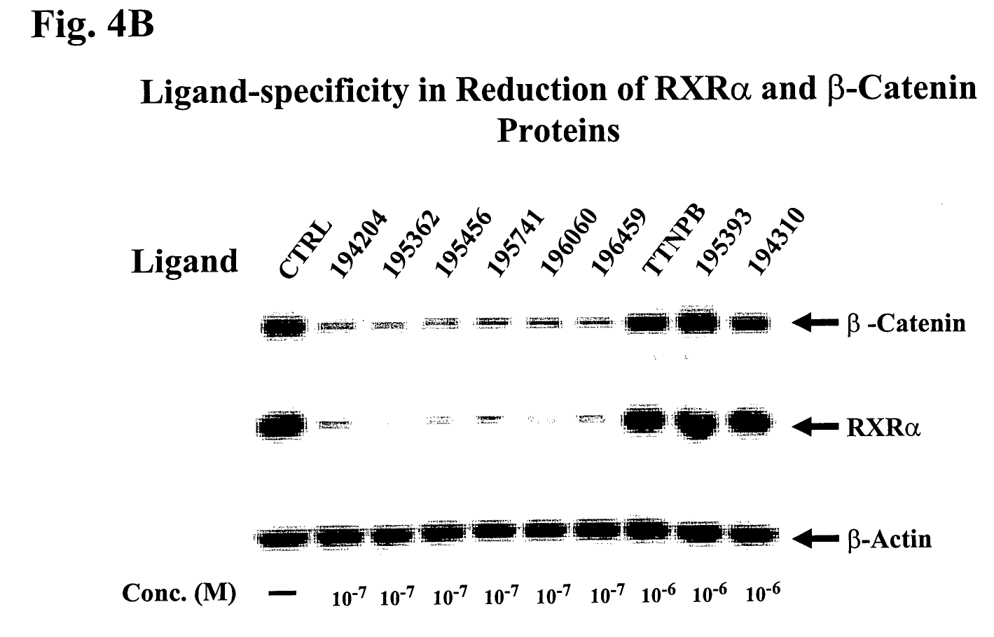

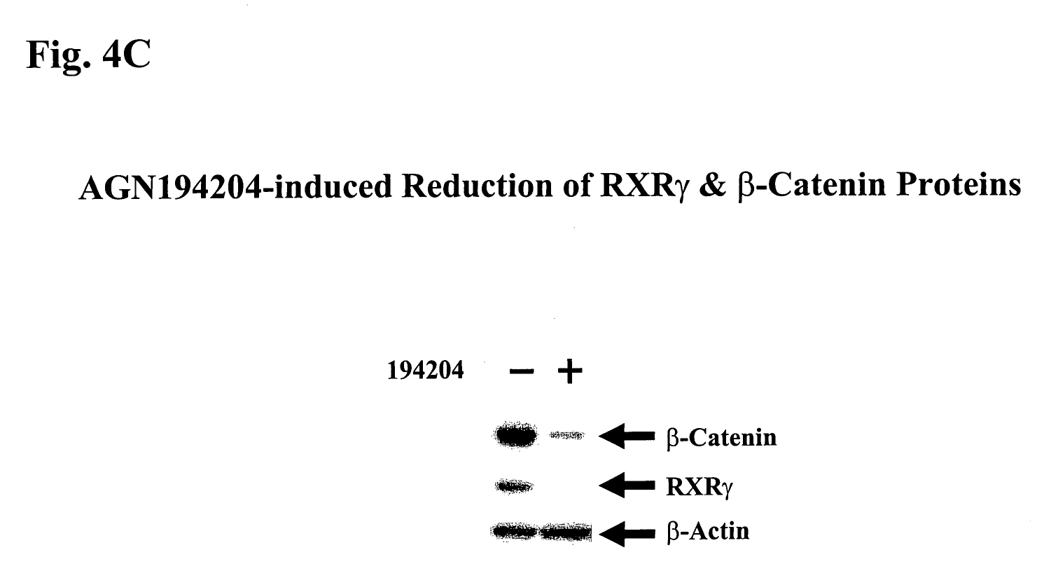

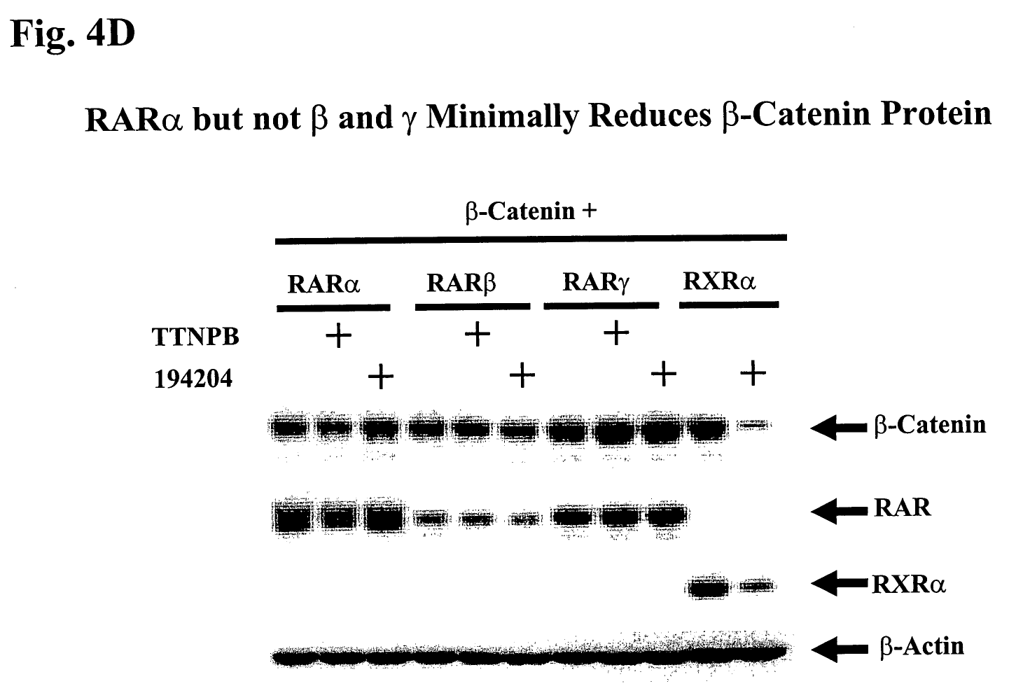

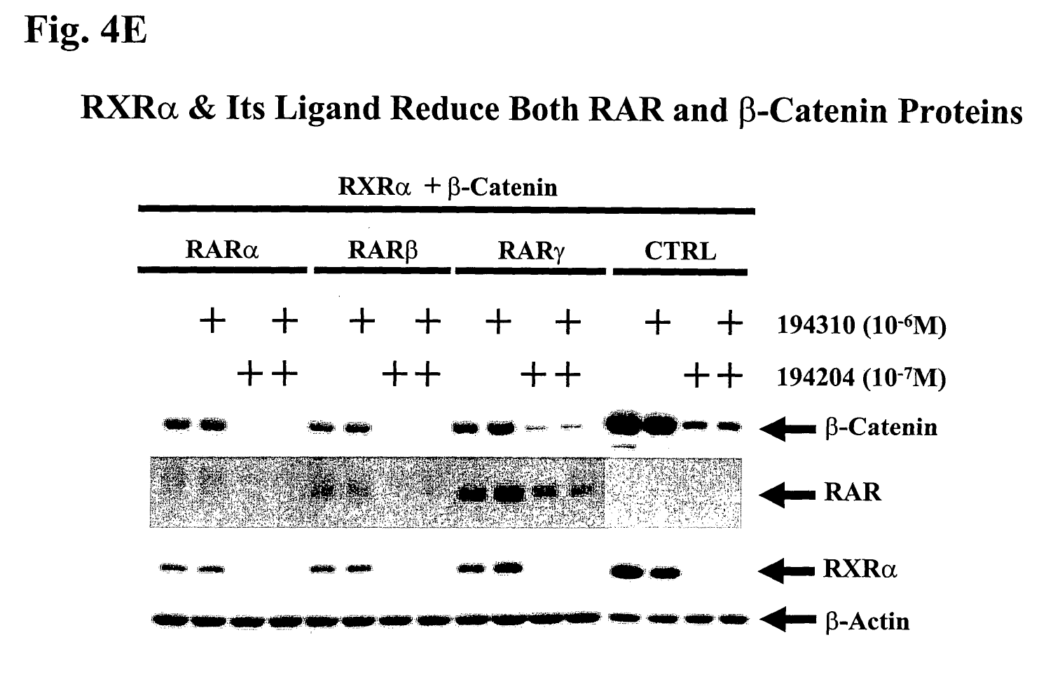

To confirm that the AGN 4204 effects are due to the direct binding of this ligand to RXR protein, HEK293 cells were transfected with both β-catenin and RXRα. Cells were then treated for 17 hours with different doses of AGN 4204 in combination with an RXR antagonist, AGN 5393. FIG. 4A shows that AGN 4204 is effective in inducing degradation of β-catenin and RXRα at a concentration of 1 nM, consistent with its binding affinity for RXRα. AGN 5393 was able to inhibit the degradation of RXRα and β-catenin. As shown in FIG. 4B, when applied alone, RXR antagonist AGN 5393, RAR agonist TTNPB and antagonist 194310 had no effect on either RAR or β-catenin stability. By contrast, various RXR agonists in addition to AGN 4204, including AGN 5362, AGN 5456, AGN 5741, AGN 6060, and AGN 6459 all stimulated the degradation of RXRα and β-catenin.

As shown in FIG. 4C, AGN 4204 is not only able to mediate the degradation of β-0catenin in the presence of RXRα, it is also able to stimulated the degradation of β-catenin through RXRγ. However, the members of the retinoic acid receptor (RAR) family have little effect on the β-catenin protein level. Overexpression of RARβ or RARγ in the presence of AGN 4204 did not result in degradation of β-catenin, although a slight decrease in both RARα and β-catenin was observed when RARα was overexpressed in response to panRAR agonist TTNPB (FIG. 4D). However, co-expression of RXRα with RARα led to RXR-ligand-dependent degradation of β-catenin, RXRα and RARα (FIG. 4E). Similar effects were observed with RXRα:RARβ and RXRα:RARγ heterodimers. The effect of AGN 4204 was not antagonized by the RAR antagonist AGN 4310, indicating that ligand binding to RXR is required and sufficient for degradation of RARs and β-catenin.

### EXAMPLE 5

### RXR Functional Domains are Required for β-Catenin Degradation

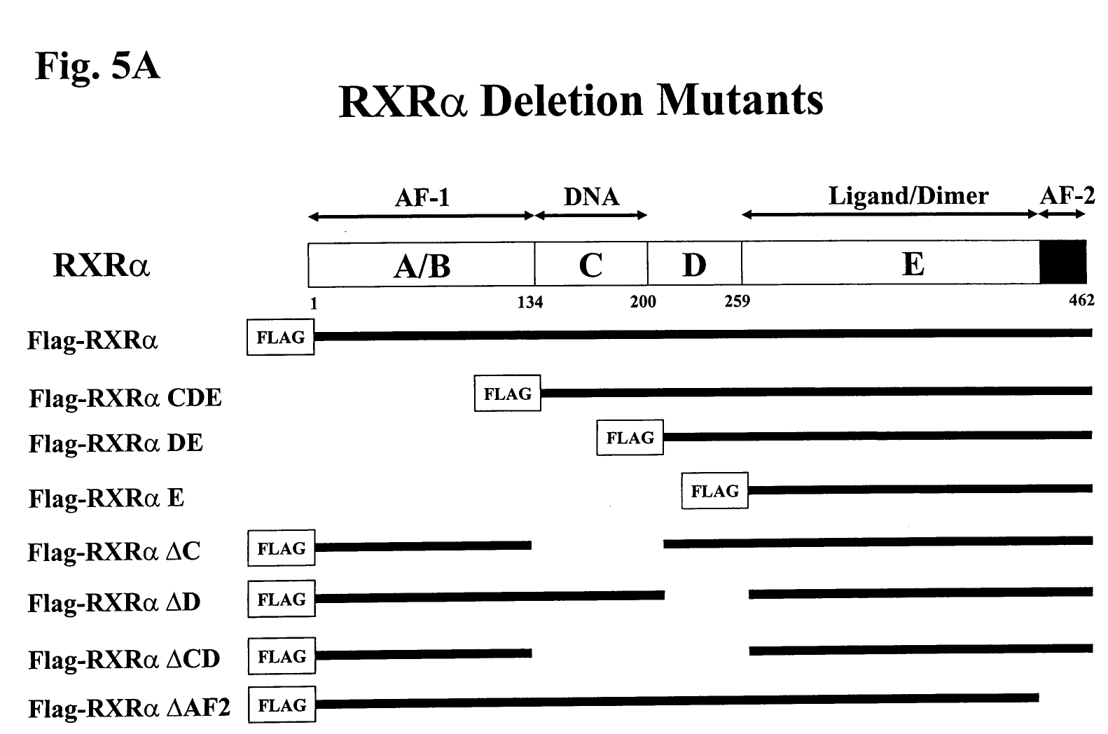

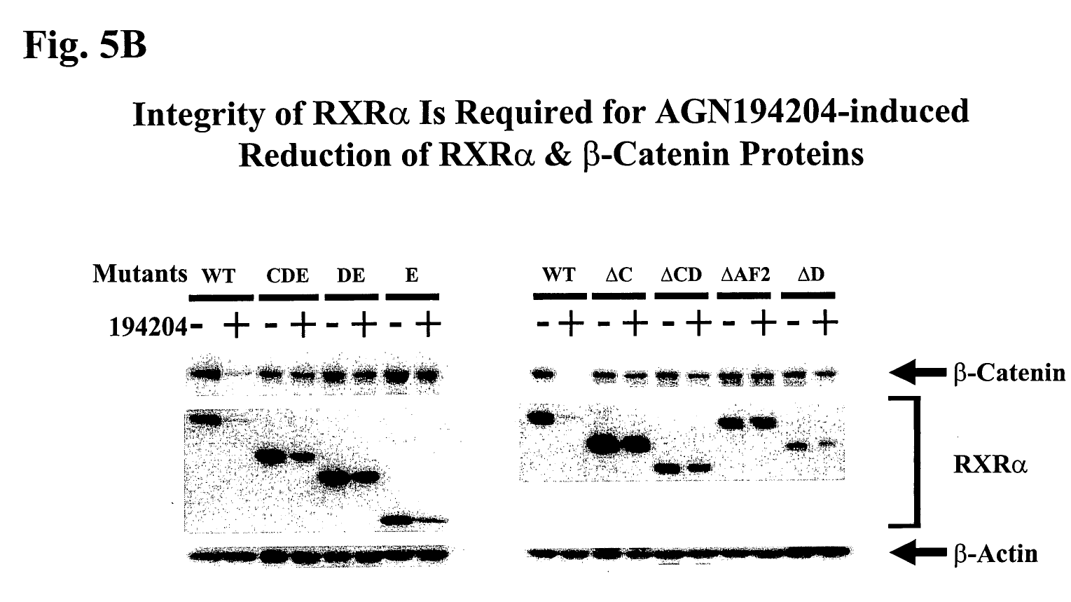

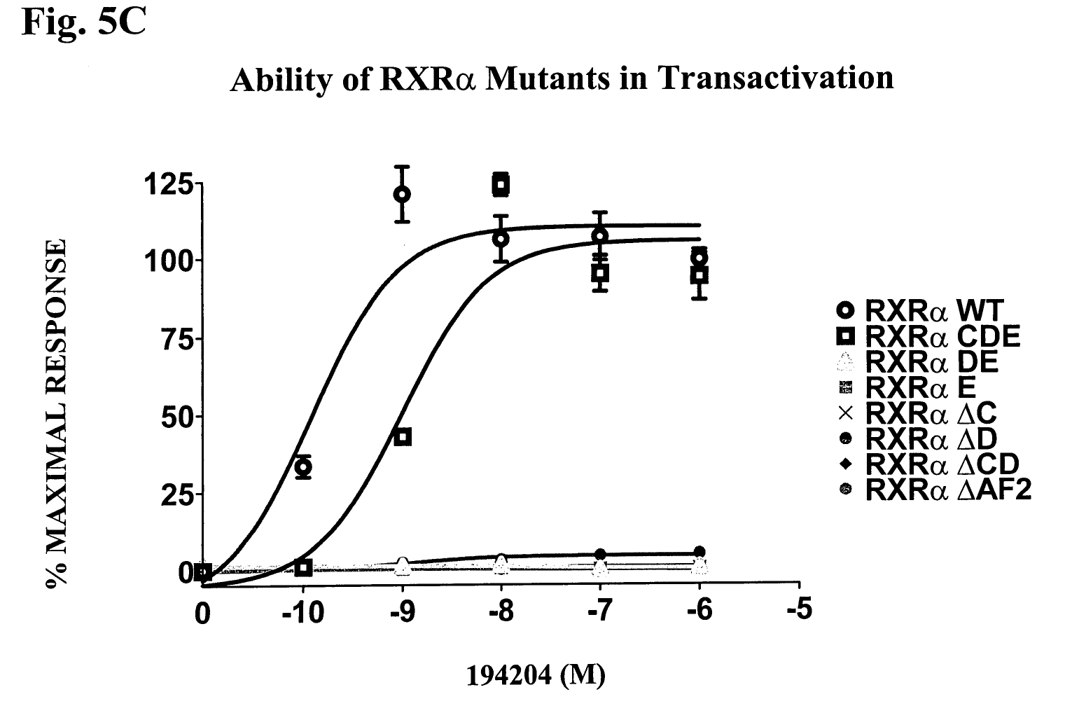

To determine the functional domains of RXRα involved in degradation of β-catenin, various deletions were introduced into the former receptor, as shown in FIG. 5A. FIG. 5B shows that deletion of different functional domains of RXRα significantly reduced its ability to mediate AGN 4204-induced α-catenin degradation. Interestingly, deletion of the N-terminal AF1 domain of RXRα abolished the ability of RXRα to induce β-catenin degradation, but not its gene transactivation activity or ability to cause its own destruction in response to AGN 4204. (FIG. 5C). The RXRα mutants having deletions of the AF-2 or DNA binding domain lost all these activities.

Thus, RXRα mutants behave differently with regard to transactivation, degradation of β-catenin, and self-destruction. Together, these observations indicate that the integrity of RXRs is essential for mediating the RXR ligand effects on β-catenin. Furthermore, the RXR ligand effects on different targets, i.e. β-catenin degradation, RXR transactivation and self-destruction, involve different function domains of the RXR molecule, suggesting different molecular mechanisms by which RXR ligands exert their effects.

### EXAMPLE 6

### Interaction of RXRα with β-Catenin

To determine whether RXRs and β-catenin interact with each other, HEK293 cells were transfected with expression vectors for RXRα and/or ΔNβ-catenin. Cells were treated with AGN 4204 for 20 min. before adding a reversible cross-linking reagent, DSP. After completion of the cross-linking reaction, cell extracts were prepared and analyzed by immunoprecipitation using antibodies against the FLAG epitope in an RXRα-FLAG fusion protein, followed by Western blotting analysis using antibodies against the V5 Tag in ΔNβ-catenin. Before loading the samples on gels, β-mercaptoethanol was added to the immunoprecipitated materials to reverse the cross-linking reaction and free the cross-linked molecules.

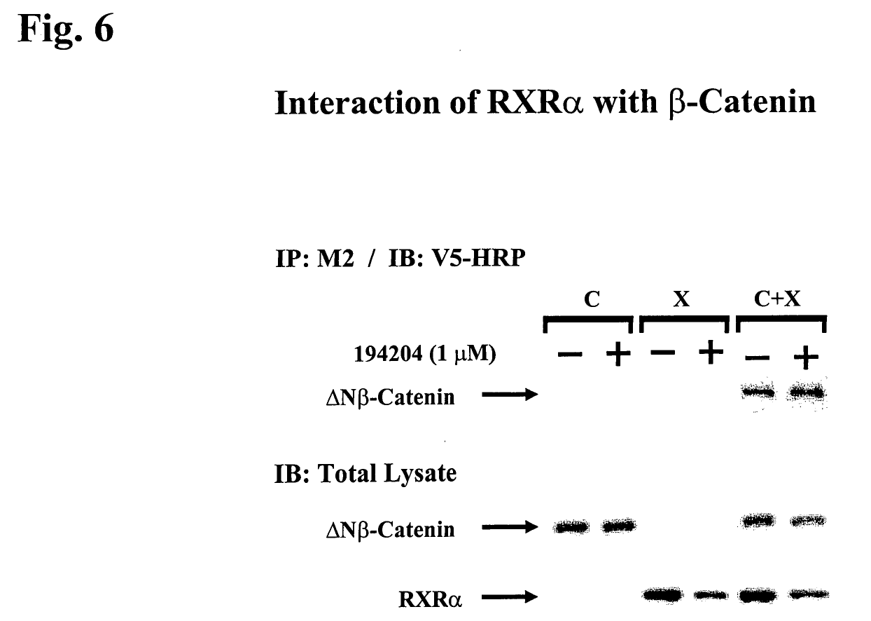

As shown in FIG. 6, ΔNβ-catenin was immunoprecipitated using antibodies raised against the FLAG epitope only in cells co-transfected with FLAG-tagged RXRα. This event was observed in cells treated with vehicle or AGN 4204. This result indicates that interaction between RXRα and β-catenin exists and is ligand-independent.

### Figure Legend

FIG. 1 Inactivation of β-catenin-mediated gene transactivation by RXR-selective retinoid AGN 4204 via RXRα. Cultured cells were transfected with 100 ng of reporter gene Topflash® together with expression vectors indicated under each graph. After transfection, cells were treated with vehicle or 0.1 μM AGN 4204 for 17 hours. (A) Data from HEK293 cells co-transfected with wild type or mutant β-catenin (200 ng) and RXRα (20 ng). (B) CAT, a stable cell line that overexpresses β-catenin (left panel). RBC, a stable cell line that overexpresses both RXRα and β-catenin. (C) SW480, a colorectal cancer cell line, was transfected with 100 ng of Topflash®. The amount of RXRα cotransfected was indicated below the X axis. Reporter activity on the Y axis is expressed as either Luciferase Unit or percentage of activity in cells transfected with Topflash® alone.

FIG. 2 RXR-dependent degradation of β-catenin protein by RXR ligand AGN 4204. (A) Western blotting analysis of HEK293 cells transfected with a combination of expression vectors for LacZ (2 μg), V5-tagged β-catenin(4 μg), and Flag-tagged RXRα (2 μg) as indicated below the gels. Cells were treated with vehicle “−” or 0.1 μM AGN 4204 “+” for 6 or 15 hours as indicated above the gels. β-galactosidase was detected by a mouse monoclonal antibody. β-catenin protein was analyzed using an HRP-conjugated mouse monoclonal antibody against the V5 tag. RXRα protein was measured using an HRP-conjugated mouse monoclonal antibody against the Flag tag. (B) Western blotting analysis of stable cell line RBC that overexpresses both β-catenin and RXRα. Cells were treated with vehicle or 0.1 μM AGN 4204 for duration indicated above the gels. (C) Western blotting analysis of stable cell line CAT that overexpresses only β-catenin. The left panel shows CAT cells transfected with the parental empty expression vector. The right panel shows CAT cells transfected with 2 μg of expression vectors for RXRα. Cells were treated with vehicle or 0.1 μM AGN 4204 for 17 hours. Endogenous and overexpressed β-catenin were detected using a rabbit polyclonal antibody as indicated by “Total” whereas overexpressed β-catenin was analyzed using a mouse monoclonal antibody against the V5 tag. Endogenous and overexpressed RXRα were detected by a rabbit polyclonal antibody against RXRα, whereas overexpressed RXRα was determined by an HRP-conjugated mouse monoclonal antibody against the Flag tag. (D) Western blotting analysis of HEK293 cells transfected with a fixed amount of β-catenin (4 μg) plus an increasing amount of RXRα as indicated at the top. Cells were treated with vehicle or 0.1 μM AGN 4204 for 17 hours. (E) Western blotting analysis of HEK293, CV-1, and Hela cells transfected with 2 μg of RXRα and 4 μg of wild type or mutant β-catenin. α. Cells were treated with vehicle or 0.1 μM AGN 4204 for 17 hours. (F) Northern blotting analysis of RBC cells using β-catenin as a probe. Cells were treated for 17 hours with vehicle or AGN 4204 at concentrations indicated above the gel.

FIG. 3 Time course of degradation of the mutant β-catenin by AGN 4204 in stable cell line RmBC. RmBC overexpresses both RXRα and the β-catenin mutant lacking the N-terminal first 50 amino acids (ΔNβ-catenin). Cells were treated with vehicle or 0.1 μM AGN 4204 for duration indicated above the gels. The levels of ΔNβ-catenin and RXRα were determined by Western blotting.

FIG. 4 Receptor and ligand specificity in degradation of β-catenin by retinoid receptors. HEK293 cells transfected with expression vectors for β-catenin (4 μg) and RXRs (2 μg) or RARs (2 μg) and treated with ligands at concentrations indicated at the top of each gel. Protein levels were detected by Western blotting as shown in this figure. β-catenin and RARs were detected by HRP-conjugated antibodies against their V5 tag. RXRs were detected by HRP-conjugated antibodies against their Flag tag. (A) shows the high potency of AGN 4204 in inducing β-catenin degradation and that this degradation activity can be inhibited by RXR antagonists 195393. (B) shows that several RXR-specific agonists (Lanes 2-7) are able to induce this degradation whereas RAR agonist TTNPB, RXR antagonist AGN195393, and RAR antagonist AGN194310 have no effects. (C) shows that RXRγ also has the ability to induce the degradation of β-catenin. (D) shows that RARs do not have significant activity in degradation of β-catenin. (E) shows that RXR and its ligand AGN 4204 are the key determinants in induction of degradation of β-catenin and RXR dimerization partners RARs. In this experiment, the amount of expression vectors for RXRα and RARs used in transfection was 1 μg.

FIG. 5 Integrity of RXRα is required for its activity in induction of β-catenin degradation by AGN 4204. (A) Diagram shows functional domains that were deleted in RXRα mutants. AF-1, transactivation function-1; DNA, DNA binding domain; Ligand, ligand binding domain; Dimer, dimerization domain; AF-2, activation function-2 domain. Solid bars indicate regions retained in the mutants. (B) Western blotting analysis of HEK293 cells transfected with β-catenin (2 μg) and RXRα mutants (4 μg). Cells were treated with vehicle or 0.1 μM AGN 4204 for 17 hours. (C) The dose-dependent effects of AGN 4204 on luciferase reporter activity in CV1 cells transfected with CRBPII-TK-Luc and RXRα deletion mutants.

FIG. 6 Interaction of β-catenin with RXRα. HEK293 cells were transfected with a combination of 8 μg of ΔNβ-catenin (C) and 4 μg of RXRα. Cells were treated with vehicle or 1 μM AGN 4204 for 15 min. before the crosslinking reaction. Cell lysates were subjected to immunoprecipitation using antibodies against the Flag tag in RXRα. Crosslinked molecules in immunoprecipitates were dissociated by reduction with β-mercaptoethanol before Western blotting analysis using HRP-conjugated antibodies against the v5 tag in ΔNβ-catenin (Top panel). Lower panel shows the direct Western blotting analysis of cell lysates reduced with β-mercaptoethanol. IP, immunoprecipitation; M2, antibody against the Flag tag in RXRα; IB, immunoblotting (Western blotting); V5-HRP, HRP-conjugated antibody against the V5 tag in ΔNβ-catenin.

We claim:

1) A method of inhibiting the proliferation of a eukaryotic cell whose growth is stimulated by β-catenin-mediated gene transcription, comprising contacting said cell with:
a) a non-endogenous source of RXR nuclear receptor protein, and
b) a therapeutically effective amount of an agonist of said RXR protein.

2) The method of claim 1 wherein said RXR agonist is not an agonist of an RAR nuclear receptor.

3) The method of claim 2 wherein said RXR protein is an RXRα protein.

4) The method of claim 1 wherein said RXR protein is expressed within said cell by an expression vector.

5) The method of claim 4 wherein said expression vector is a viral expression vector.

6) The method of claim 5 wherein said expression vector is selected from the group consisting of an adenovirus-derived expression vector, an adeno associated virus-derived expression vector and a retrovirus-derived expression vector.

7) The method of claim 6 wherein said expression vector is an adenovirus-derived expression vector.

8) The method of claim 4 in which said expression vector is injected into said cell.

9) The method of claim 1 in which said cell is a colon cell and said non-endogenous source of RXR protein is provided to said cell by means of oral or rectal administration.

10) The method of claim 9 in which said RXR ligand is contacted with said cell by systemic administration.

11) The method of claim 1 wherein said cell is a cancer cell.

12) The method of claim 11 wherein said cancer cell is a colon cancer cell.

13) A method for determining whether a test compound is an RXR agonist comprising administering said test compound to a cell which expresses RXR and β-catenin, and determining whether β-catenin is degraded in response to the addition of said test compound, wherein the degradation of said β-catenin indicates that said test compound is an RXR agonist.


SEQUENCE LISTING (24 sequences)

SEQ ID NO: 1 - DNA, 30 bp, Artificial Sequence
  PCR Sequence
agggatccaaccatgaatcctgaggaagag

SEQ ID NO: 2 - DNA, 30 bp, Artificial Sequence
  PCR Sequence
agtctagattacaggtcagtatcaaaccag

SEQ ID NO: 3 - DNA, 32 bp, Artificial Sequence
  PCR Sequence
aggaattcatggacaccaaacatttcctgccg

SEQ ID NO: 4 - DNA, 32 bp, Artificial Sequence
  PCR Sequence
agctgcagctaagtcatttggtgcggcggctc

SEQ ID NO: 5 - protein, 8 aa, Artificial Sequence
  FLAG tag
DYKDDDDK

SEQ ID NO: 6 - DNA, 29 bp, Artificial Sequence
  PCR Sequence
aggaattctgcgccatctgcggggaccgc

SEQ ID NO: 7 - DNA, 33 bp, Artificial Sequence
  PCR Sequence
agggtaccctaagtcatttggtgcggcgcctcc

SEQ ID NO: 8 - DNA, 35 bp, Artificial Sequence
  PCR Sequence
aggaattcaagcgggaagccgtgcaggaggagcgg

SEQ ID NO: 9 - DNA, 33 bp, Artificial Sequence; identical to SEQ ID NO: 7
  PCR Sequence

SEQ ID NO: 10 - DNA, 29 bp, Artificial Sequence
  PCR Sequence
aggaattctcgccgaacgaccctgtcacc

SEQ ID NO: 11 - DNA, 33 bp, Artificial Sequence; identical to SEQ ID NO: 7
  PCR Sequence

SEQ ID NO: 12 - DNA, 32 bp, Artificial Sequence; identical to SEQ ID NO: 3
  PCR Sequence

SEQ ID NO: 13 - DNA, 30 bp, Artificial Sequence
  PCR Sequence
aggaattcgatgtgcttggtgaaggaagcc

SEQ ID NO: 14 - DNA, 30 bp, Artificial Sequence
  PCR Sequence
aggaattccatgcccatggccaggcacttc

SEQ ID NO: 15 - DNA, 32 bp, Artificial Sequence; identical to SEQ ID NO: 3
  PCR Sequence

SEQ ID NO: 16 - DNA, 31 bp, Artificial Sequence
  PCR Sequence
gggtaccctagatgagcttgaagaagaagag

SEQ ID NO: 17 - DNA, 29 bp, Artificial Sequence
  PCR Sequence
aggaattctgcgccatctgcggggaccgc

SEQ ID NO: 18 - DNA, 32 bp, Artificial Sequence
  PCR Sequence
agggtaccctagatgagcttgaagaagaagag

SEQ ID NO: 19 - DNA, 35 bp, Artificial Sequence; identical to SEQ ID NO: 8
  PCR Sequence

SEQ ID NO: 20 - DNA, 32 bp, Artificial Sequence; identical to SEQ ID NO: 18
  PCR Sequence

SEQ ID NO: 21 - DNA, 29 bp, Artificial Sequence
  PCR Sequence
aggaattctcgccgaacgaccctgtcacc

SEQ ID NO: 22 - DNA, 32 bp, Artificial Sequence; identical to SEQ ID NO: 18
  PCR Sequence

SEQ ID NO: 23 - DNA, 32 bp, Artificial Sequence
  PCR Sequence
aggaattcatgtatggaaattattctcacttc

SEQ ID NO: 24 - DNA, 33 bp, Artificial Sequence
  PCR Sequence
agggtacctcaggtgatctgcagcggggtctcc